(19) United States
(12) Patent Application Publication
(10) Pub. No.: US 2009/0123385 A1
(43) Pub. Date: May 14, 2009

(54) PREPARATION OF DISPERSIONS OF PARTICLES FOR USE AS CONTRAST AGENTS IN ULTRASOUND IMAGING

(75) Inventors: Marcel Rene Bohmer, Eindhoven (NL); Holger Gruell, Eindhoven (NL); Freddy Roozeboom, Eindhoven (NL); Antonius Lucien Adrianus Maria Kemmeren, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/577,821

(22) PCT Filed: Oct. 29, 2005
(86) PCT No.: PCT/IB05/53486

§ 371 Date: Apr. 24, 2007

(30) Foreign Application Priority Data
Oct. 29, 2004 (EP) 04105413.1

(51) Int. Cl. A61B 8/06; B01F 13/00

(52) U.S. Cl. 424/9.52; 366/162.4

(57) ABSTRACT

A method of making a suspension of particles in a first fluid of a size suitable for responding to ultrasound, by forcing a second fluid (2) through an array of pores (30) into the first fluid (1), the pores being of substantially uniform diameter, and the pressure of the second fluid and a flow rate of the first fluid across the pores being arranged so that shear forces at the pores cause the second fluid to be suspended as substantially monodisperse particles in the first fluid. This can enable a more monodisperse suspension. The array of pores can be an etched silicon array. The suspension can be used as a contrast agent, or can be an emulsion to make droplets of a precursor material, which can be formed into capsules with a core and a shell. A liquid core can be converted to a gas to provide hollow shells.

This invention relates to methods of making dispersions of colloidal systems, including suspensions of particles or emulsions, methods of manufacturing dispersions of particles including colloidal systems, suspensions or emulsions as well as capsules and apparatus for the carrying out such methods.

Ultrasound is the most widely used method in imaging-based medical diagnostics. To date ultrasound imaging is almost exclusively based on acquisition of morphological data. Image quality and therefore, diagnostic value can be improved significantly by application of intravenously injected contrast agents. Such agents are also used in other imaging based diagnostics such as computer tomography (CT) and magnetic resonance imaging (MRI).

Presently available ultrasound contrast agents comprise hollow particles, bubbles or “so-called” gas-filled liposomes that interact with sound very effectively. Such contrast agents usually have a fairly wide size distribution. The interaction with the sound field depends on the particle size, and, therefore, there will be quite some variation in acoustic behaviour of the contrast agent particles. A further method of using bubble contrast agents is harmonic imaging in which the harmonic signals emitted from oscillating bubbles are detected. Such signals will be sharp and located at a well-defined frequency. This may, in principle, make it possible to distinguish between free flowing contrast agent and contrast agent in a narrow capillary or attached to a vessel wall or blood clot, as this will change the resonance frequency.

The application of contrast agents in targeted imaging, i.e. molecular imaging of a disease by use of a contrast agent having a bio-targeting agent, and in drug delivery is emerging, see for instance Dayton et al. in Molecular Imaging, 3 (2004), pp. 125-134, Lanza et al., Prog. Cardiovascular Dis. 44 (2001), pp. 13-31, and as well as the activities of Imarx, see www.imarx.com and, for instance, U.S. Pat. No. 6,146,657. The above applications strongly depend on the physical and chemical properties of the contrast agents, for instance the size distribution, the mechanical modulus of the shell, and the biodegradability. Currently available contrast agents are poorly defined in size and have highly variable mechanical properties. It is known to make such contrast agents more monodisperse by filtering using sieves for example. U.S. Pat. No. 6,193,951 discloses size distributions before and after filtering, e.g. example 15. This is filtration of the complete emulsion/suspension. As a rule of thumb every fractionation method leads a large losses and many desirable particles are also removed. Filtration processes on the complete emulsion or suspension are selective for the particle or emulsion droplet size they are not selective for other parameters that are important for the characteristics in an external field, such as ultrasound. One of those parameters is the thickness of the shell of the contrast agent. In the preparation of liposomes filtration is used often, extrusion is a filtration-based process, which is carried out the complete solution.

Another use of ultrasound may be in localized and targeted therapy. Ultrasound-induced activation of particles may be applied to release drugs at a well-defined location. For effective control of the release, focused ultrasound irradiation of particles with well-defined size and mechanical properties is required. This is for instance noted by Cherry et al., Phys. Med. Biol. 49, R13 (2004).

Ultrasound contrast agents for intravenous use have been used in ultrasound imaging for a number of years. They are based on the use of gas bubbles and to slow down the disappearance of the bubbles, the bubbles have a shell. The shell consists of proteins, lipids, and/or biodegradable polymers. The size of the bubbles is about the size of a red blood cell or slightly smaller and they are effective in extremely small amounts. There are many types of ultrasound contrast agents, for an overview see Klibanov, “Ultrasound contrast agents: Development of the field and current Status” in Contrast Agents II, ed. W. Krause, Springer, 2002, pages 74-103.

In ultrasound imaging, as well as in other medical imaging techniques, attempts have been made to enhance the functionality for instance by targeting the imaging agent to a specific, diseased area. As ultrasound contrast agents will normally stay in the blood pool, cardiovascular diseases, such as vulnerable plaque, thrombosis and damaged endothelial cells can be targeted with ultrasound contrast agents bearing specific bio-targeting agents such as antibodies, fragments thereof or peptide sequences. Angiogenesis is also a process that can be followed in more detail using contrast agents. Apart from the visualization of new vessels, their surface characteristics are different and can depend on the pathology, for instance the presence of a tumor. Finally, the vasculature near a tumor is often leaky, allowing contrast agents to escape from the circulation. A useful reference discussing many aspect of ultrasound and the use of contrast agents is: “Contrast-enhanced Ultrasound of Liver Diseases”, Solbiati et al., Springer 2003.

It is also known that ultrasound contrast agents can be combined with therapeutic agents. The contrast agents are loaded with drugs, which are released upon insonification. A local high dose can be provided which can enable opportunities for treatment of for instance thrombosis or local vasodilators.

An object of the invention is to provide methods of making dispersions such as, suspensions of particles or emulsions, as well as capsules and apparatus for carrying out such methods. The present invention has the advantage of being able to provide contrast agents with a narrow size distribution that give a more uniform response to the sound field during ultrasound diagnosis, therapy and imaging e.g. harmonic imaging. Another advantage is that there are in principle few or no losses of particles which are within the size range which can be used. Another advantage is that not only the size but also the composition is very uniform, leading to a well-defined shell thickness giving more uniform response to the sound field during ultrasound diagnosis, therapy and imaging e.g. harmonic imaging.

According to a first aspect, the invention provides a method of making a dispersion, for instance an emulsion in a first fluid, the particles having a size suitable for responding to ultrasound or other diagnostic tools, by forcing a second fluid through one or more pores or nozzles, into the first fluid, the nozzles or pores being of substantially uniform diameter, a flow parameter such as pressure of the second fluid being such so as to cause the formation of second fluid suspended as substantially monodisperse droplets in the first fluid. The pressure of the second fluid may be controlled to thereby assist the formation of second fluid suspended as substantially monodisperse droplets in the first fluid. A flow rate of the first fluid across the one or more nozzles or pores may be controlled so that shear forces at the one or more nozzles or pores assist the formation of the droplets. The dispersion may be in any suitable form, e.g. a colloidal system such as an emulsion. The emulsion may be converted to a suspension in a post-treatment step. Both the first and second fluids may be liquids.

In one aspect of the present invention submerged inkjet printing of the second fluid into the first fluid is carried out, a flow of the first fluid is then not necessary. For example, if the second fluid is a liquid that is purged through a capillary, it will break up in droplets of equal size. This is the case with submerged inkjetting by which a second liquid is forced into a first liquid. An additional optional feature is imposing a frequency or vibration to the inkjet ink chamber to cause the meniscus at the nozzle exit to vibrate and hence detach. Capillary instabilities break up the second fluid into droplets. The pores may be part of a membrane of controlled porosity, or be microchannels or an SPG (Shirasu-porous glass) membrane, for example. Flow of the first liquid across these pores can give more monodisperse emulsions. In this case the flow of the first liquid is advantageous as it exerts a force on the droplet being formed and controls the break-off. Flow of the first fluid is not an essential feature of the present invention, forcing a second fluid through a surface with a well defined porosity in to a first fluid without transverse flow of the first fluid is still within the scope of the present invention and can give a narrower distribution than other means of filtration or sizing.

The above method can enable a more monodisperse dispersion to be created. This can be useful not only for ultrasound but other imaging techniques, and for drug delivery using ultrasound or other techniques.

An additional feature of the present invention is that the array of pores or nozzles comprises an etched array in a suitable substrate, e.g. in a semiconductor material such as silicon.

Another such additional feature is the pores being orientated at an angle, not perpendicular to the flow of the first fluid. This can be advantageous for the “snap-off” of partly formed droplets of the second fluid, e.g. liquid by the first liquid, e.g. liquid because the formed droplet will have a region with higher curvature.

Another additional feature is the dispersion comprising a contrast agent suitable for diagnostic imaging.

Another such additional feature is the pores or nozzles having a coating to alter a wetting property.

Another such additional feature is a further processing step to convert the emulsion droplets to shells or bubbles filled with a gas. This will generally generate a suspension of particles, e.g. microbubbles or microballoons.

Another additional feature is that the particles in the dispersion comprise a polymer or lipids, such as phospholipids, glycolipids or cholesterol.

Another such additional feature is the array of pores or nozzles is in a first substrate and is supported by a second substrate, e.g. of a different material.

Another aspect provides apparatus for carrying out the method. In particular the present invention provides an apparatus making a dispersion of particles in a first fluid of a size suitable for responding to ultrasound or other diagnostic tools, comprising:

means for forcing a second fluid through an array of nozzles or pores into the first fluid, the nozzles or pores being of substantially uniform diameter, a flow parameter of the second fluid being such that second fluid is suspended as substantially monodisperse droplets in the first fluid. The apparatus may also include first controlling means for controlling a flow parameter of the second fluid to assist the second fluid being suspended as substantially monodisperse droplets in the first fluid

A second controlling means may be provided for controlling a flow rate of the first fluid across the nozzles or pores so that shear forces at the nozzles or pores assist the second fluid to be suspended as substantially monodisperse particles in the first fluid.

A further feature is a method of manufacturing capsules of a size suitable for responding to ultrasound, from substantially monodisperse droplets of a precursor material according to the above. The method includes forming the droplets into capsules with a core and a shell, and then modifying the core. The substantially monodisperse droplets can be an emulsion.

This can enable capsules with a more consistent size. A further advantage is that the composition of the particles is controlled, as all the material initially present in a drop of the second liquid will end up in a particle. This relates directly to providing shell thickness control.

An additional feature of the present invention is that the particles have a core comprising a liquid and the modifying step, e.g. freeze drying, comprising converting the core to a gas.

Another such additional feature is the droplets having a hydrophobic phase e.g. an oil phase, and the modifying step comprising selective solvent removal from the hydrophobic phase.

Another such additional feature is the droplets originating from the second fluid being a solution of a biodegradable polymer, such as poly-(lactic-acid), poly-(glycolic-acid) poly-caprolacton, poly-(alkyl-cyanoacylates) and poly-(amino-acids) and copolymers thereof in a polar organic solvent, such as a halogenated solvent, esters and ethers, including ethylene-glycol, and isopropyl-acetate, dimethylformamide and N-methyl-pyrolidon or acetone or dichloromethane or dichloroethane. To this solution a non-polar, non-solvent for the biodegradable polymer is added, examples are alkanes such as cyclo-octane and dodecane and fluorinated liquids.

Another additional feature is the step of dissolving the polar solvent from the droplets into the first liquid. This can be achieved by choosing a polar solvent that has a small but limited solubility in the first liquid and can subsequently be removed from the first liquid by any suitable means, e.g. evaporation or extraction. Means to promote or reduce the solubility of the polar solvent in the first liquid include post treatment modifying the temperature or changing the ionic strength.

Another inherent or additional step is a phase separation of the biodegradable polymer and non-polar solvent, resulting in a shell of biodegradable polymer, and a core of non-polar solvent. This can occur together with the removal of the polar solvent because at a certain percentage of removal the polymer will precipitate and form the shell.

Another additional step is lyophilization to remove the non-polar solvent. The alkane preferably does not have too high a molecular weight, e.g. preferably the alkane has a molecular weight as low or lower than dodecane. Cyclo-octane is a preferred solvent as it is solid at 15° C., and should give less deformation during fast initial freezing.

Another additional feature is the capsules having a diameter of less than 20 μm and more than 1 μm, preferably less than 6 microns and a standard deviation of less than 15%, preferably smaller than 10%, e.g. 7% or lower with respect to the mean particle diameter.

Another aspect of the invention provides additional apparatus for carrying out the method. In particular the present invention provides an apparatus for manufacturing capsules of a size suitable for responding to ultrasound from substantially monodisperse droplets of a precursor material, comprising:

means for forming the droplets into capsules with a core and a shell, and means for modifying the core.

Another additional feature is means for dissolving the polar solvent from the droplets into the first liquid. This can be achieved by choosing a polar solvent that has a small but limited solubility in the first liquid and can subsequently be removed from the first liquid by any suitable means, e.g. evaporation or extraction. Means to promote or reduce the solubility of the polar solvent in the first liquid include post treatment modifying the temperature or changing the ionic strength.

Another inherent or additional feature is means for a phase separation of the biodegradable polymer and non-polar solvent, resulting in a shell of biodegradable polymer, and a core of non-polar solvent. This can occur together with the removal of the polar solvent because at a certain percentage of removal the polymer will precipitate and form the shell.

Another additional feature is means for lyophilization to remove the non-polar solvent.

Any of the additional features can be combined together and combined with any of the aspects. Other advantages will be apparent to those skilled in the art, especially over other prior art. Numerous variations and modifications can be made without departing from the claims of the present invention. Therefore, it should be clearly understood that the form of the present invention is illustrative only and is not intended to limit the scope of the claims.

How the present invention may be put into effect will now be described by way of example with reference to the appended drawings, in which:

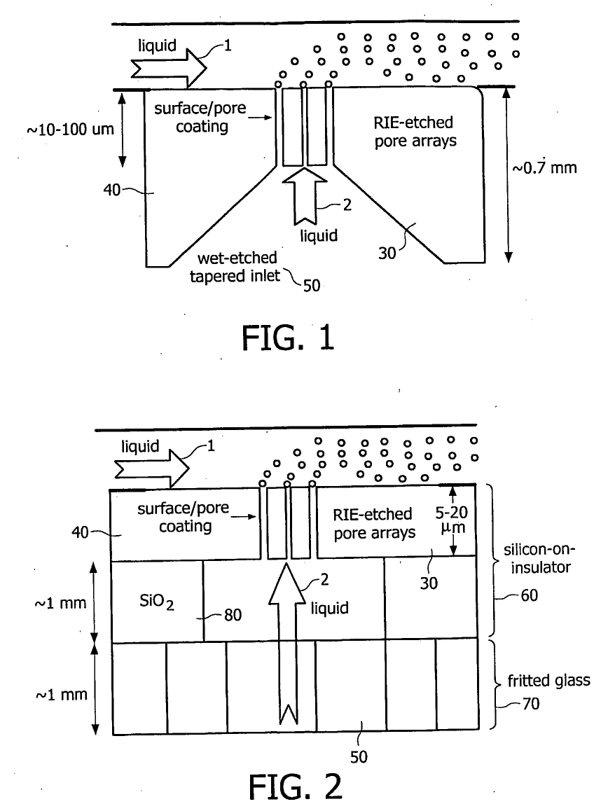

FIG. 1 shows apparatus according to a first embodiment of the present invention,

FIG. 2 shows apparatus according to an embodiment of the present invention,

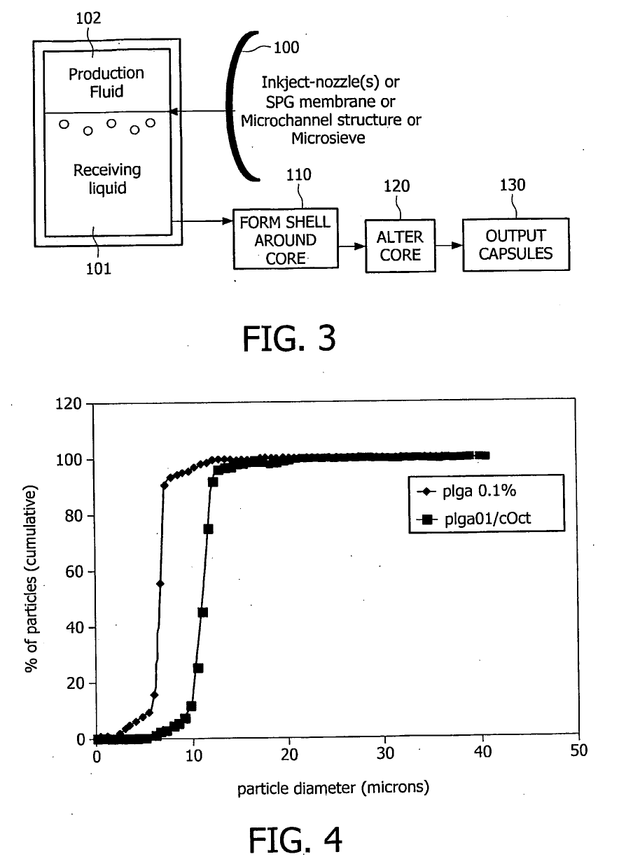

FIG. 3 shows schematically another embodiment of the present invention, and

FIG. 4 shows a graph of percentage of particles being a given diameter.

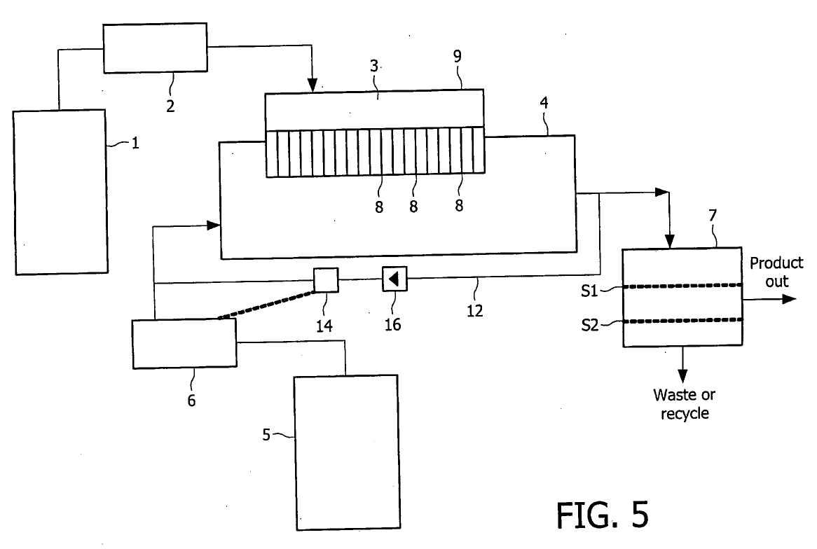

FIG. 5 shows a system according to an embodiment of the present invention,

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

The term “comprising”, used in the claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

The present invention provides a method of making a dispersion of particles in a first fluid, the particles having a size suitable for responding to ultrasound or other diagnostic tools, by forcing a second fluid through one or more nozzles or pores, e.g. through an array of nozzles or pores into the first fluid. A suitable droplet size is preferably about 4 micrometer in diameter, e.g. preferably smaller than 10 and larger than 1 micrometer. This procedure creates droplets.

The final form of the droplets may be as a suspension or an emulsion. An emulsion is the suspension of a hydrophobic phase (or a hydrophilic phase) in a hydrophilic phase (or hydrophobic phase, respectively). In an emulsion the two phases can be immiscible in each other or may be partly immiscible. In the latter case substances which have both hydrophobic and hydrophilic properties such as surfactants or lipids or even an pegylated lipid may be used as emulsifiers, i.e. they determine the boundary between the hydrophobic and hydrophilic phases.

Generally, a suspension is a solid/liquid system while an emulsion is a liquid/liquid system. In embodiments of the present invention which use two liquids, an emulsion is made first and optionally this emulsion is converted into a suspension. This is, for example, the case for polymeric particles and can be extended to capsules with polymer shells. The emulsion can comprise particles having a lipid shell encapsulating a liquid. In this case droplets stabilized by lipids are formed.

The emulsifier can be added to the first liquid and may be, for example a water soluble polymer or a surfactant. An suitable polymer is poly-(vinyl-alcohol), preferable with a degree of hydrolysis less than 90% and more than 70%. Other polymeric stabilizers are poly-(vinyl-pyrolidone), copolymers of poly-(ethylene-oxide) and poly-(propylene oxide). Poly-amino-acids can also be used as a stabilizer. Surfactants can be used as well, preferably surfacants with an ethylene-oxide polar group. A stabilizer can also be added to the second fluid. Block-copolymers of the listed biodegradable polymers, where the additional block is a poly-ethylene oxide are excellent stabilizers. The poly-ethylene oxide block is often in the molecular weight range of 2000 Dalton. The latter option is particular advantageous as no excess stabilizer has to be removed in subsequent processing steps. Pegylation can also be used to influence the biodistribution.

When the contrast agents are used in the vasculature of the human or animal body the environment is usually hydrophilic. In this regard the first fluid is preferably aqueous. The particles will belong to the hydrophobic phase.

Any suitable method of creating droplets having a uniform size by forcing a second fluid through a porous surface into a first fluid can be used with the present invention. General techniques of making droplets by a non-impacting method have been developed by the printing industry. Examples are given in the book “Principles of Nonimpact printing”, Jerome L. Johnson, 2^{nd} edition, Palatino Press, 1998 and include inkjet printing, such as impulse inkjet printing or piezoelectric inkjet printing. The printing operation is used to generate particles and is preferably carried out submerged beneath the surface of the first fluid.

Deflection inkjet printing can be used in one embodiment of the present invention to provide very accurate size distributions. Usually, in deflection inkjet printing the droplets are deflected in the air to print a dot or not onto paper. In accordance with the an embodiment of the present invention droplets are deflected when in the first fluid. This can be useful in removing satellites which are very small droplets emitted by the ink jet head under some circumstances. The size of the droplets is measured at the outlet side of the inkjet nozzle, e.g. using an optical method and droplets are deflected or trapped which do not meet a specific volume size, e.g. the satellites. The optical method can include a strobe light to freeze the motion of the particles and allow optical measurement or can include other methods such as change of output of an optical sensor caused by the particle obscuring a part of the incident light. The measuring technique should give a measure of the size of the particle. By this technique very uniform drop sizes can be obtained. Unused material can be recycled.

Alternatively, fractionation after the inkjetting step can be carried out to remove the satellites with a high yield as satellites have a distinctly different size. Moreover, this fractionation does not have an effect on the uniformity of the composition of the particles, which for ultrasound contrast agents is for instance uniformity in shell thickness.

The wording “flow of the second liquid” refers to a macroscopic description of the process, on a microscopic scale the liquid is brought into the first liquid drop by drop, e.g. a droplets starts to form on the outlet of the channel or capillary, fills to a critical size and then breaks off by itself simply by the flow of the second liquid or by the imposition of energy to detach the droplet, e.g. a vibration or modulation of the pressure in the second fluid, e.g. at a vibration at a high frequency. Per droplet the flow is not necessarily constant, it is rather a nucleation and growth process which can be forced. To force the nucleation a pressure is required. Hence the second fluid is forced through the nozzles or pores The pressure needed to achieve nucleation is related to the interfacial tension (γ) between the first liquid and the second liquid, In the presence of stabilizers, fairly low values can be obtained, lower than 30 mN/m or even lower than 20 mN/m. If the interfacial tension is 30 mN/m and the pore diameter is 2 μm (d) the Laplace pressure that equals 4γ/d=60 kPa. Apart from the Laplace pressure a pressure drop over the pores or channels is present, which depends on the shape of the capillaries. Pressures of more than 10⁵ Pa can be used.

The techniques described above can use mechanical or electromechanical pulses to generate droplets. The pulses do not need to be sufficient to generate free standing droplets. Due to the flow of first fluid passed across the openings of the nozzles, second liquid which has formed a convex meniscus by a smaller pulse can be dragged away by the flow of the first fluid at a time when the meniscus has not reached sufficient size for the droplet to break free if the flow of first fluid were not present.

The present invention also includes the use of continuous flow of the second fluid to generate droplets. In this case, due to the flow of first fluid passed the opening of the nozzles, second liquid which has formed a convex meniscus by constant flow can be dragged away by the flow of the first fluid at a time when the meniscus has not reached sufficient size for the droplet to break free if the flow of first fluid were not present. To exert a reasonable force on the droplet, a fairly high flow rate at the interface is preferably present. The shear stress near the wall surrounding the pores determines the force on the droplet being formed, typical values are higher than 1 Pa, preferably higher than 10 Pa are preferred. It has been found that values above 30 Pa are not necessary for alpha-alumina membranes with a pore diameter of 0.8 μm,—see Schröder and Schubert, Colloids and Surface A, physicochemical and engineering aspects 152, (1999), 103-109.

The nozzles or pores used to generate the droplets are usually substantially uniform in diameter, and a flow parameter, e.g. the pressure, of the second fluid and a flow rate of the first fluid across the nozzles or pores is preferably arranged so that shear forces at the nozzles or pores assist the second fluid to be suspended as substantially monodisperse droplets in the first fluid.

A first embodiment of the invention, illustrated in FIG. 1 shows apparatus for creating a suspension of monodisperse droplets which can be used directly as contrast agents, or can be used to create a precursor from which such agents can be formed. To manufacture the pores or nozzles, anisotropic dry or wet etching of a substrate can be used to form very regular arrays of pores, e.g. in a semiconductor substrate such as monocrystalline silicon or silicon-on-insulator wafers, or in any other suitable substrate, e.g. plastic, glass, quartz or a metal such as copper. An anisotropic etch can be used to generate the pores. The pores may also be made by any other suitable technique, e.g. using laser pulse drilling. Alternatively commercially available arrays of pores can be used, e.g. inkjet printheads. The first embodiment of FIG. 1 a suspending pore array etched in Si, by RIE etching of front-side wafer, and subsequent KOH etching of the wafer backside is shown (not to scale).

The pores for any of the embodiments of the present invention have preferably diameters in between 0.5 and 5 micrometer for liquid/liquid systems, a pitch of 10-20 micrometer and depths from 10 to over 25 micrometer in any suitable substrate, e.g. in monocrystalline silicon or silicon-on-insulator wafers or glass or alumina or metal substrates. Smaller pores may also be used, e.g. nucleopore membranes can be used.

The narrow pores serve as fine channels through which a liquid can be pressed. This liquid will be referred to as the second fluid. This fluid enters on the backside and leaves at the frontside, where it flows into another liquid, which will be called the first fluid. The first fluid flows across the pores, i.e. flows transversely across the pore openings. There, due to the specific shear forces characteristic for the combination of liquids or liquids, the exiting second fluid is suspended as highly monodisperse droplets. Such highly monodisperse droplets can be used directly or converted effectively as contrast agents for ultrasound imaging. The dimensions and shape of these pore arrays can be further tuned to tailor the droplet size. Also the pore arrays may be fully or locally coated in order to change the wetting properties of the pores or pore outlets and their periphery in order to further tailor size and shape of the droplets. The coating is shown with reference number 40 in FIGS. 1 and 2.

In addition, the present invention provides methods for the production of suspensions of highly monodisperse particles, e.g. capsules consisting of a shell of a controlled chemical composition and filled with a gas. These can be used for completely new applications of ultrasound, namely for early disease diagnostics by molecular imaging and targeted therapy. By using pore arrays, e.g. pores etched in a substrate such as silicon of sufficient thickness and mechanical strength, a microdevice is provided that can be made that produces monodisperse suspensions.

The present invention provides contrast agents with improved physical and chemical properties, for instance an improved size distribution, an improved mechanical modulus of the shell, and an improved biodegradability as phagocytosis depends on size and surface properties. While the use as normal contrast agents is not that demanding, the present invention allows molecular imaging and drug release that require designated particles with a narrow size distribution, e.g. monodisperse and a well-defined shell elasticity.

The highly monodisperse emulsions can be used as contrast agents directly, for instance if a perfuorocarbon liquid is incorporated, or can be further processed to yield microbubbles or microballoons with a shell, e.g. of a polymer or a phospholipid. Alternatively such pores, nozzles or microchannels may be used to create small gas bubbles of, for instance, a perfluorocarbon gas and if such bubbles are led through a solution containing phospholipids, for instance, gas filled liposomes or microbubbles are generated and can be used as ultrasound contrast agent directly. This can help enable new options in early disease diagnostics by molecular imaging and targeted therapy. To create microbubbles directly, a smaller pore size may be required. Contrary to the liquid/liquid system no shrinkage has to occur, and contrary to the liquid/liquid systems no problems with the pressure occur to press the second fluid into the first fluid. Nucleopore membranes with pore diameters of 200 nm are well suited for this.

A method to manufacture a regular injection pore array 30 according to an embodiment of the present invention uses a substrate such as silicon into which an array of fine pores having a diameter typically a few micrometer with special shapes is formed, e.g. cylindrical, triangular, square, rectangular, hexagonal etc. These shapes can promote the shear of the second liquid. An anisotropic etching technique can be used such as an RIE-etch to depths of several tens of micrometers in a conventional Si (100)-wafer. The bulk of the wafer backside etching is then done by wet-etching, e.g. isotropic, using an etchant such as KOH where the typical shape along the Si-(111) crystallographic planes automatically serves as a tapered inlet 50 for the second fluid (liquid 2).

The wafer backside may be further mechanically strengthened by bonding the porous Si-wafer onto a support of a robust material with macro-openings corresponding to the tapered inlet openings of the Si-wafer.

The device described can be used to obtain monodispersed emulsion droplets containing the precursors of the microbubbles to be formed, for instance using, as the second fluid, a solution of a biodegradable polymer, such as poly-(lactic-acid), poly-(glycolic-acid) poly-caprolacton, poly-(alkyl-cyanoacylates) and poly-(amino-acids) and copolymers thereof in a polar organic solvent, such as a halogenated solvent, esters and ethers, including ethylene-glycol, and isopropyl-acetate, dimethylformamide and N-methyl-pyrolidon or acetone, dichlormethane. To this solution a non-polar, non-solvent for the biodegradable polymer is added, examples are alkanes such as cyclo-octane and dodecane and fluorinated liquids. After formation of the emulsion the non-polar solvent will slowly dissolve in the continuous phase, leading to a shrinkage of the emulsion droplets and a phase separation of the biodegradable polymer around the polar solvent. Optionally, lyophilization can be used to remove any of the polar solvent yielding hollow capsules with a shell of biodegradable polymer.

In a specific embodiment of the present invention monodispersed emulsion droplets containing the precursors of the microbubbles to be formed, are formed using a solution of poly-lactic-co-glycolic acid (PLGA) in dichloromethane and dodecane. After formation of the emulsion the polar solvent, e.g. dichloromethane will slowly dissolve in the continuous phase, leading to a shrinkage of the emulsion droplets and a phase separation of the PGLA and dodecane resulting in dodecane filled capsules. By lyophilization, the dodecane is removed yielding hollow capsules with a PLGA shell. The polymers and solvents used can be varied.

Another embodiment of the present invention shown in FIG. 2 shows a suspending pore array etched in Si-on-Insulator, 60 by RIE etching of the front-side Si-part and subsequent HF etching of the back-side glass part SiO₂ (not to scale). In this Figure the etched SOI wafer is further strengthened by fritted glass 70.

Similarly to the first RIE etching step described in the first embodiment an array of fine pores with the same dimensions and cross-sectional shapes can be etched into so-called silicon-on-insulator (SOI) substrates. When using the so-called Bosch process with SF₆/C₄F₈ chemistry the RIE process will selectively stop upon reaching the Si—SiO₂ interface. The SiO₂ underneath the pore arrays can be etched, e.g. in HF, such that large openings are created allowing for liquid 2 to enter into the fine pore arrays. If desired the entire SOI wafer can be bonded onto a further mechanically strengthening support such as a fritted glass support, that it permeable to liquid 2. The bonding can be by thermal compression, or any other suitable technique.

The devices described can be used to generate monodisperse emulsion droplets which can be further processed as described above. Further strengthening of the substrate can be realized by adding baffles to the top side, oriented such that the direction of the flow is not blocked.

Well-defined ultrasound contrast agents made in the way described above can make it possible to obtain superior images, even for very small blood vessels. Furthermore, applications in ultrasound imaging, especially targeted ultrasound imaging as well as in therapy, especially targeted and localized therapy are provided by the present invention. Both applications rely on the availability of well-defined particles. As an example of targeted ultrasound imaging, the present invention can be used in any specific pathology like vulnerable arterial plaque, which plays a major role in acute cardiovascular disease, in blood vessels. Ultrasound-assisted local drug delivery is a second and very important application, which is enabled by the proposed particle manufacturing methods. In accordance with this embodiment micro-bubbles made in accordance with the present invention are loaded with drugs. The drugs can be dissolved in for instance on oil phase, such as paraffin or a natural oil that is not removed by lyophilization that can be added to the second liquid. Relevant drugs are for instance anti-cancer drugs as paclitaxel and deoxyrubicin. These are introduced into a patient and the capsules are opened at the desired location in the body by ultrasound disruption. Local release of chemotherapy drugs can greatly minimize the occurrence of undesired side effects of these highly toxic chemicals.

A third embodiment relates to post treatments to form contrast agents with a narrow size distribution that can be prepared using emulsification methods where a hydrophobic phase, such as an “oil” phase is added drop-by-drop to a hydrophilic continuous phase, such as an “aqueous” continuous phase and all these drops have similar size and shell properties. Subsequently by selective solvent removal from the “oil-phase” the monodisperse contrast agent is prepared. Suitable techniques are as described above, e.g. ink-jet printing, microchannel emulsification, filtration through shirasu-porous glass and filtration to microsieves e.g. those etched in silicon.

A notable feature of the embodiment is that an emulsification technique is used that makes droplets all have the same size and composition containing the precursor material for the contrast agent, e.g. as a hydrophobic phase, by forcing this into another phase, e.g. hydrophilic phase such as an aqueous phase. A further advantage is that the composition of the particles is controlled, as all the material initially present in a drop of the second liquid will end up in a particle. By appropriate processing the droplets will be transformed into capsules with a liquid core and, subsequently the liquid is removed to yield hollow capsules that can be filled with a chosen gas. Because each emulsion droplet is converted into a single capsule, and the emulsion droplets start from the same size, the capsules formed have a narrow size distribution, the same shell thickness and shell properties that determine the acoustic behaviour.

Techniques to achieve such monodisperse emulsion droplets include any of the methods described above, e.g. submerged inkjet, microchannel emulsification, SPG (Shirasu Porous Glass) membrane emulsification and microsieve filtration. The drop-by-drop methods allow preparing particles with a well-defined size, shell thickness and composition, leading to an ultrasound contrast agent that gives uniform acoustic behaviour.

Schematically an example of the third embodiment is shown in FIG. 3. A production fluid 102, i.e. the second fluid is brought into a receiving liquid 101, i.e. the first fluid, in droplets that are all of the same size and suitable to finally yield particles of the desired dimensions for an ultrasound contrast agent. A suitable size is about 4 micrometer in diameter, e.g. preferably smaller than 10 and larger than 1 micrometer. The drawing shows further processing steps for the emulsion droplets of forming a shell 110. For example the solvent is removed and then the core 120 is altered, e.g. by changing phase from liquid to gas. Finally the capsules, e.g. hollow capsules are output. The interface 100 between the reservoirs of the production fluid 102 and the receiving liquid 101 contains any suitable nozzles or pores, e.g. inkjet-nozzles, a shirasu porous glass membrane, microporous alumina, a microchannel structure or a microsieve. In all cases a controlled flow of the second fluid is needed to achieve a well-controlled size of the emulsion droplets. Hence the present invention includes control of a flow parameter of the second fluid, e.g. pressure.

An example procedure is given below:

A 0.1% solution of PLGA and 0.3% of cyclo-octane in dichloroethane was prepared and ink-jetted into a 0.1% PVA 40/88 solution at a frequency of 14 kHz using a 50 μm nozzle. Dichloroethane was evaporated, the sample was washed with water previously saturated with cyclo-octane, and freeze-dried. Capsules with a diameter of 11.2 μm with a standard deviation of 1.6 μm were formed, as quantified using image analysis of optical microscopy pictures. The size distribution is indicated in FIG. 4, where the size distribution of solid PLGA particles prepared according to the same recipe without the cyclo-octane is given for the sake of comparison. Capsules had a smooth surface and contained one single cavity as deduced from SEM pictures.

Another way of producing small polymer spheres is to use an SPG membrane. These have been used to produce polymer spheres of similar materials (see Kaminski et al., presented at the 5th Int. Conference on the Scientific and Clinical Applications of Magnetic Carriers, Lyon, France, May, 2004). This demonstrates the compatibility with the chosen materials. SPG membranes have also been used to prepare capsules, e.g. of non-biodegradable polymers in the size range of 4 micrometers (see LY Chu et al, J. Colloid Interface Science, 265, 187-196, 2003), FIG. 6 in this paper gives an impression of the size distribution that can be achieved.

FIG. 5 is a schematic diagram of an apparatus for producing particles in accordance with an embodiment of the present invention. A source of the second fluid, e.g. a liquid, is shown with reference number 1. The liquid in the source 1 is fed by gravity or by a pump (not shown) to a head 3, which comprises nozzles or pores 8 and is located in a container 9. The present invention includes that each nozzle or group of nozzles 8 has a separate source of second fluid and each nozzle or group of nozzles is controlled separately. Alternatively, all of the nozzles may be fed from a single source and by controlled by a single controller. A flow parameter of the second fluid is controlled by a controller 2 which may be a pressure controller. The controller 2 may be an open loop controller or it may be a closed loop controller which receives an input from a pressure sensor (not shown) in the second fluid loop and controls the flow of second fluid, e.g. by controlling the pump or a valve to meter second fluid to the head 3 at the correct pressure/flow. The first fluid is provided in a source 5 and is fed to another input of chamber 9 by means of gravity or via a pump (not shown). In some embodiments of the present invention the feed of the first fluid generates a flow of fluid 1 across the front ends of the nozzles 8. The flow of the first fluid is controlled by a controller 6. The controller 6 may be an open loop controller or it may be a closed loop controller which receives an input from a flow sensor (not shown) in the first fluid loop and controls the flow of the first fluid, e.g. by controlling the pump or a valve to meter first fluid to the container 9 at the correct pressure/flow. The particles are collected in chamber 7. Further sizing of the particles may be performed, e.g. by an oversize sieve S1 which holds back oversized particles and/or an undersized sieve S2 which allows too small particles to be flushed from the system. Instead of the sieves S1 and S2 any other fractionation based on particle density may be used. This can be used to remove satellites. Another method of fractionation is to make use of the fact that the flotation velocity depends on the particle size.

Optionally a bypass 12 can be provided which allows the continuous phase, i.e. the first fluid to pass the porous surface more than one. The flow may be controlled by a once way flow device 16 and by a valve 14 which may be controlled by the controller 6 or may be controlled separately. In this way, the first fluid can collect more emulsion droplets because the number of passes of the first fluid past the membrane can be varied independently.)

The nozzles 8 can be any of the nozzles described in embodiments of the present invention. The nozzles or pores are of substantially uniform diameter, and the controllers control a flow parameter of the second fluid and a flow rate of the first fluid across the nozzles or pores so that shear forces at the nozzles or pores cause the second fluid to be suspended as substantially monodisperse particles in the first fluid.

The flow of second fluid to the nozzles may be determined by mechanical or electromechanical pulses to generate droplets. The pulses do not need to be sufficient to generate free standing droplets. Due to the flow of first fluid passed the opening of the nozzles, second liquid which has formed a convex meniscus by a smaller pulse can be dragged away by the flow of the first fluid at a time when the meniscus has not reached sufficient size for the droplet to break free if the flow of first fluid were not present. The present invention also includes the controlling the second fluid in a continuous flow to generate droplets. In this case, due to the flow of first fluid passed the opening of the nozzles, second liquid which has formed a convex meniscus by constant flow can be dragged away by the flow of the first fluid at a time when the meniscus has not reached sufficient size for the droplet to break free if the flow of first fluid were not present.

The apparatus may be used to produce capsules and may generate cores of material and include additional ancillary equipment, e.g. as shown in FIG. 5, namely means for forming a shell around the core, means to alter the core and means for outputting the capsules and described with reference to FIG. 3.

Applications of the particles include ultrasound contrast agents, especially targeted ultrasound contrast agents. Various ultrasound applications can benefit from the better acoustic properties of contrast agents with a well-defined size distribution and consistent shell properties in accordance with the present invention. Monodisperse ultrasound contrast agents have many advantages. As harmonic peaks are more distinct compared to polydisperse agents, the contrast to tissue ratio improves. This advantage can be exploited further if a mixture of two monodisperse contrast agents with a distinctly different size is used: the presence of two harmonic peaks proves that one is looking at the contrast agent. The performance of pressure measurements using ultrasound contrast would become possible: The resonance frequency of a bubble is to a good approximation given by the Minnaert frequency. The resonance frequency in rad/s is given by:

$${ω}_{0} = \frac{1}{R} \sqrt{\frac{3 p}{ρ}} \qquad ( 1 )$$

where p is the pressure, R the radius of the bubble and ρ the density of the fluid. For an adiabatic case the 3 in the numerator has to be replaced by 3γ, with γ being the polytropic gas coefficient (e.g. 1.4 for air). For an air bubble with radius of 2 μm, in water, under atmospheric conditions, ω₀=8.7·10⁶ rad/s, which is 1.37 MHz.

Again using a mixture of two distinct sizes would improve the quality of the pressure measurement.

For targeted contrast agents a tight size distribution can lead to a discrimination between adhered and non-adhered contrast agent. For a contrast agent with a wide size distribution this has been shown by Dayton et al. Molecular Imaging vol 3 no 2 April 2004, p 125-134. They study the accumulation of contrast agent targeted to αᵥβ₃ integrins and observe a shift in the echo spectra to lower frequencies for adhering contrast agent. A shift in the direction observed by Dayton et al is predicted by Scott in J. F. Scott “Singular perturbation theory applied to the collective oscillation of gas bubbles in a liquid”, J. Fluid Mech. 113, 487-511 (1981). This theory is based on potential flow calculations but can reasonably Well extrapolated to smaller bubbles as well. A function is disclosed that describes the decrease of the resonance frequency close to a wall, or for the similar case of two equally sized bubbles. When the bubble touches the wall a resonance frequency of 0.83 ω₀ was determined. For high surface coverage an additional decrease can be expected, Duineveld, J. Acoust. Soc. Am. 99, 622-624, 1996, demonstrated the effect of a decrease of the resonance frequency of two equally sized bubbles experimentally. If monodisperse targeted contrast agents are used, the distinction between bound and unbound contrast agent is expected to become much evident compared to the result by Dayton et al. The use of monodisperse contrast agents allows the shift to be studied more quantitatively and potentially extract clinical relevant information. Also in this case a mixture of distinctively different sizes could be employed targeted to different markers, for instance VEGF and αᵥβ₃ integrins.

For drug delivery a better control of the size distribution using the proposed preparation methods has the advantage that the amount of drug incorporated is also well controlled. Drug release can therefore be quantified. With uniform shell properties of the agents, release by cavitation is also under better control than with a polydisperse sample.

1. A method of making a dispersion in a first fluid, particles in the dispersion having a size suitable for responding to ultrasound or other diagnostic tools, by forcing a second fluid through one or more pores or nozzles into the first fluid, the nozzles or pores being of substantially uniform diameter, a flow parameter such as pressure of the second fluid being controlled to thereby cause the formation of second fluid being suspended as substantially monodisperse droplets in the first fluid.

2. A method according to claim 1, wherein the first fluid is flowed across the one or more nozzles or pores so that shear forces at the one or more nozzles or pores assist the formation of the dispersion.

3. The method of claim 1, the droplet forming step using one or more steps selected from using a submerged ink-jet printing head, microchannel emulsification, filtration through shirasu-porous glass and filtration through a microsieve.

4. The method of claim 1, wherein the array of nozzles or pores are in an etched silicon substrate.

5. The method of claim 2, the nozzles or pores being orientated at an angle, not perpendicular to the flow of the first fluid.

6. The method of claim 1, the nozzles or pores having a coating to alter a wetting property.

7. The method of claim 1, the dispersion comprising a contrast agent suitable for diagnostic imaging.

8. The method of claim 1, the droplets comprising a polymer or a phospholipid.

9. The method of claim 1, the array of nozzles or pores being in a first substrate and supported by a second substrate of a different material than the first substrate.

10. The method of claim 1 for the manufacture of capsules of a size suitable for responding to ultrasound, the method further comprising:
creating substantially monodisperse droplets of a precursor material,
forming the droplets into capsules with a core and a shell, and then modifying the core.

11. The method of claim 10, and a further processing step of converting the droplets to shells filled with a gas.

12. The method of claim 11, the core modifying step comprises converting the core to a gas.

13. The method of claim 10, the droplets being in a hydrophobic phase and comprising a solvent, and the core modifying step comprising selective solvent removal from the droplets.

14. The method of claim 10, the droplets comprising a solution of a biodegradable polymer in a polar solvent and with an added amount of a non-polar solvent.

15. The method of claim 14, and a step of dissolving or removing the polar solvent.

16. The method of claim 14 and a step of phase separation of the biodegradable polymer and the non-polar solvent, resulting in a shell of biodegradable polymer, and a core of non-polar solvent.

17. The method of claim 14 and the step of lyophilization to remove the non-polar solvent.

18. The method of claim 10, the capsules having an average diameter of less than 20 μm and a standard deviation of less than 15% of the mean diameter.

19. The method of claim 10 the capsules having an average diameter of less than 6 μm and a standard deviation of less than 15% of the mean diameter.

20. An apparatus for making a dispersion in a first fluid, particles of the dispersion being of a size suitable for responding to ultrasound or other diagnostic tools, comprising:
means for forcing a second fluid through an array of nozzles or pores into the first fluid, the nozzles or pores being of substantially uniform diameter, and first controlling means for controlling a flow parameter of the second fluid so that second fluid is suspended as substantially monodisperse droplets in the first fluid.

21. The apparatus of claim 20, further comprising second controlling means for controlling a flow rate of the first fluid across the nozzles or pores so that shear forces at the nozzles or pores assist the second fluid to be suspended as substantially monodisperse droplets in the first fluid.

22. The apparatus of claim 20, for manufacturing capsules of a size suitable for responding to ultrasound from monodisperse droplets of a precursor material, further comprising:
means for forming the droplets into capsules with a core and a shell, and
means for modifying the core.
